(19) United States
(12) Patent Application Publication
(10) Pub. No.: US 2005/0129790 A1
(43) Pub. Date: Jun. 16, 2005

(54) Polyphenol-containing stem and vine extracts and methods of use

(76) Inventors: John D. Folts, Madison, WI (US); Dhanansayan Shanmuganayagam, Madison, WI (US); Paul R. Hutson, Verona, WI (US)

Correspondence Address: FOLEY & LARDNER, 150 EAST GILMAN STREET, P.O. BOX 1497, MADISON, WI, 53701-1497, US

(21) Appl. No.: 10/990,930

(22) Filed: Nov. 17, 2004

Related U.S. Application Data
(60) Provisional application No. 60/523,078, filed on Nov. 17, 2003.

(51) Int. Cl. A61K 35/78; A61K 31/353

(52) U.S. Cl. 424/766; 514/456

(57) ABSTRACT

The present invention relates to extracts of polyphenolic-containing stems and/or vines. There are provided methods for extracting polyphenols from polyphenolic-containing stems and vines as well as extracts produced by these methods. In other aspects, the invention provides dietary supplements, nutraceutical and pharmaceutical compositions containing the stem and/or vine extracts and methods of using the extracts to prevent or treat coronary artery disease and other diseases.

### CROSS-REFERENCES TO RELATED APPLICATIONS

This application claims priority to U.S. Provisional Application No. 60/523,078, filed Nov. 17, 2003, the entire contents of which are incorporated by reference herein.

### STATEMENT OF GOVERNMENT INTERESTS

This invention was made with United States government support awarded by the following agencies: USDA/CSREES 97-36200-5789. The United States has certain rights in this invention.

### FIELD OF THE INVENTION

The present invention relates to methods for producing polyphenol-containing extracts from stems and vines of plants containing the same, and in particular, stems and vines of grapes. The invention further relates to compositions containing such extracts, and methods of use thereof to prevent or treat coronary artery disease and other diseases.

### BACKGROUND OF THE INVENTION

In spite of recent advances in diagnosis and treatment, coronary artery disease, cerebral artery disease, peripheral artery disease and heart failure continue to be the leading causes of mortality and morbidity in the United States. More than one million people per year have heart attacks in the United States and one-third of them die from their first heart attack. Thus, there is considerable interest in the prevention of coronary and cerebral artery disease.

Polyphenolic extracts from grapes have attracted widespread attention because of their cardio-vascular-protective properties. Epidemiological studies examined the protective effect of red wine consumption in reducing cardiovascular diseases. Numerous studies have since shown that such cardio-protective effects are strongly correlated to the presence of polyphenols in wine. Other studies indicate that purple grape juice has antioxidant and anti-platelet activity comparable to that of red wine.

While red wine and purple grape juice are readily available, not everyone can take advantage of their cardio-protective properties. For example, many people cannot or choose not to drink alcoholic beverages such as red wine. Similarly, others choose not to drink grape juice because of its high levels of sugar. For such people, dietary supplements containing grape extracts are a convenient source of polyphenolic compounds. The grape extracts found in dietary supplements are typically made from the grape skins and/or seeds remaining after the berries have been pressed to extract their juice for wine making, fruit juice production or other purposes. These extracts typically have modest levels of the desired polyphenolic compounds and result in low potency dietary supplements. On the other hand, processing the grape juice or wine itself leads to a higher potency polyphenolic extract that is also fairly expensive. Thus, there exists a need in the art for an inexpensive source of polyphenols that can be readily processed to give high potency dietary supplements. The present invention fulfills this need and provides further advantages that will become apparent from the following description.

### SUMMARY OF THE INVENTION

In accordance with various aspects of the present invention, there are provided methods for extracting polyphenolic compounds from stems and vines which contain the same. Other aspects of the invention provide compositions including dietary supplements, and pharmaceutical and nutraceutical compositions containing the polyphenolic extracts from stems and vines. In accordance with yet other aspects of the present invention, there are provided methods of preventing or treating coronary artery disease, cerebro vascular disease, peripheral-vascular disease, atherosclerosis, atherosclerosis related diseases, and heart failure. Thus, there are provided high potency polyphenolic-containing extracts at relatively low cost for use in dietary supplements, nutraceutical compositions, and pharmaceutical compositions.

### BRIEF DESCRIPTION OF THE DRAWINGS

FIG. 1 illustrates the inhibition of the platelet aggregation response produced by calculated dry weight equivalents of grape seed extract, grape skin extract and grape stem extract.

FIG. 2 illustrates a chart recording arterial blood pressure and coronary artery blood flow over time in a canine cyclic flow reduction model of thrombosis.

FIG. 3 shows arterial blood pressure and coronary artery blood flow over time in a cyclic flow reduction model after administration of extract from 173 mg fresh stem/kg body weight.

FIG. 4 shows arterial blood pressure and carotid artery blood flow in a cynomologous monkey CFR model before and after infusion of extract from 135 mg fresh stem/kg body weight.

FIG. 5 shows the effect of grape stem extract administration on the platelet aggregation response in the canine CFR model.

FIG. 6 shows inhibition of in vitro LDL oxidation by seed, skin and stem when compared to baseline control.

FIG. 7 shows the efficacy of grape stem extract of the present invention combined with grape seed and/or grape skin extracts in inhibiting platelet aggregation.

### DETAILED DESCRIPTION OF THE INVENTION

It has been discovered that the stems and vines of certain plants, such as grape stems and vines, are rich in polyphenolic compounds and may be extracted to provide useful compositions in the prevention and treatment of heart diseases. In one aspect there are provided methods of extracting polyphenolic compounds from stems and vines, the methods including treating a mixture comprising polyphenolic-containing stems, vines, or combinations thereof with one or more solvents to produce an extract containing polyphenolic compounds. In some embodiments, a mixture of acetone and water is used as the extraction solvent. The extract can be enriched in polyphenolic compounds by chromatography.

It should be understood that the exact amount of stems and vines in the mixture being extracted is not critical to the method, but that as the amount of stems and/or vines increases in the mixture, the amount of polyphenolic compounds recovered increases. Typically, the stems, vines, or combinations thereof comprise at least a third, at least 50%, at least 70%, or at least 90% of the mixture by weight prior to treatment with solvent. In some embodiments, the mixture comprises at least 90% stems by weight prior to treatment with solvent. The mixture to be extracted may also include polyphenolic-containing leaves.

As employed herein, the phrase “polyphenolic compounds” refers to polyphenols having two or more phenol groups or masked phenol groups (e.g. as ketones). Thus polyphenolic compounds include flavonoids such as flavones, flavonols, anthocyanidins, anthocyanins, dihyroflavones, dihydroflavonols, flavans, chalcones, isoflavones as well as other substances including procyanidins, gallic acids, caftaric acids, phenolic acids and the like. In particular, the present methods enrich the extracts in polygalloyl poly-flavan-3-ols. By “enriched” it is meant that the extract contains more of the stated component as a percentage of the extract than prior to chromatography.

The present methods optionally include processing the mixture to increase the surface area of the stems and vines prior to treating with one or more solvents. As contemplated herein, such processing may include grinding, mashing, tearing, or cutting the stems, vines, or combinations thereof prior to extraction. Frequently it is advantageous to freeze, chemically or mechanically, and then grind the frozen stems and vines prior to treating with one or more solvents. The stems and vines are conveniently frozen by exposure to liquid nitrogen or a dry ice/acetone bath, or in a freezer. The stems and vines may also be freeze dried (lyophilized) prior to processing. In some embodiments, the stems and/or vines are processed to a fine powder before extraction.

Extractions of the mixture of stems and/or vines may be performed using techniques known in the art. Typically the methods include treating the mixture one, two, or more times with one or more solvents and includes agitating the stems and/or vines with the solvent manually or mechanically (e.g., by stirring, shaking or sonication). The extractions may be performed using one or more solvents selected from water, alcohols, esters, sulfoxides, ketones, or mixtures of any two or more thereof. In particular, useful solvents include water, methanol, ethanol, ethyl acetate, t-butyl acetate, dimethylsulfoxide, acetone, methylethylketone, or mixtures of any two or more thereof.

Extractions of stems and vines with mixtures of water and water-miscible solvents generally give good to excellent results. Thus, extractions can be performed with one or more solvents selected from water/acetone, water/methanol, water/ethanol, or water/DMSO. Typically acetone and water mixtures are used. The amount of acetone in such mixtures can range from about 50% to about 99% by volume, including from about 60% to about 95%, with about 80% being an especially suitable amount. The water in aqueous extraction solvents can be acidic (i.e., pH<7), basic (i.e., pH>7) or neutral and has a pH of about 1 to about 14. Typically, the pH is anywhere from about 2 to about 7 or about 8. Extractions may be performed multiple times with multiple pH ranges to achieve an optimal extract. Solvent may be removed from the combined extracts in part or in whole to yield the concentrated or dried extract or a concentrated solution of the extract. Fibrous solids and other particulate matter may be removed from the extract by centrifugation and/or filtration such as microfiltration or ultrafiltration according to methods known to the skilled artisan.

Chromatography of the stem/vine extract enhances the recovery of polyphenolic compounds beneficial in the prevention and treatment of heart disease. While a wide variety of chromatographic methods may be used including adsorption, ion, and permeation chromatography, a dual method such as adsorption/permeation chromatography is especially suitable. For example, the extract may be chromatographed on hydroxypropylated dextran such as Sephadex LH-20 using an isocratic solvent system or a solvent gradient. A solvent gradient that is either a step gradient or a continuous gradient can be used. The gradient can run from a polar solvent such as water to a less polar solvent such as water and a water-miscible organic solvent such as acetone. While the water is typically about pH 7, it is within skill of the practitioner to adjust the pH up or down as necessary according to need. The ratio of water to organic solvent in the latter mixture may be adjusted as appropriate according to need as understood by those of skill in the art. Typically, a mixture of from about 1% to about 99% acetone by volume is used. However, mixtures having about 25% to about 99%, about 50% to about 99%, about 65% to about 95%, or about 80% acetone by volume can be used. The fractions containing polyphenolic compounds can be concentrated to remove acetone or both acetone and water, and may be lyophilized to produce a powder rich in polyphenolic compounds such as polygalloyl flavan-3-ols.

The present methods may be utilized with any polyphenolic-containing stems or vines such as grape, cranberry, or blueberry stems or vines. Suitable genera include Vitis (grape), Vaccinium (blueberry), Malus (apple), Musa (banana), Prunus (cherry/peach), Fragaria (strawberries), Rubus (raspberry and blackberry), Sambucus (elderberry), and combinations thereof. Typically the stems or vines are grape stems or vines, including but not limited to Vitis viniferous, rotundifolia, aestivalis, californica, labrusca, riparia, and rupestris. Exemplary cultivars include but are not limited to Aleatico, Alicante Bouschet, Aligote, Alvarelhao, Aramon, Baco blanc (22A), Burger, Cabernet franc, Cabernet, Sauvignon, Calzin, Carignane, Charbono, Chardonnay (including, e.g., CH 01, CH 02, CH Dijon), Chasselas dore, Chenin blanc, Clairette blanche, Concord, Early Burgundy, Emerald Riesling, Feher Szagos, Femao Pires, Flora, French Colombard, Fresia, Furnint, Gamay, Gewurztraminer, Grand noir, Gray Riesling, Green Hungarian, Green Veltliner, Grenache, Grillo, Helena, Inzolia, Lagrein, Lambrusco de Salamino, Malbec, Malvasia bianca, Mataro, Melon, Merlot, Meunier, Mission, Montua de Pilas, Muscadelle du Bordelais, Muscat blanc, Muscat Ottonel, Muscat Saint-Vallier, Nebbiolo, Nebbiolo fino, Nebbiolo Lampia, Orange Muscat, Palomino, Pedro Ximenes, Petit Bouschet, Petite Sirah, Peverella, Pinot noir, Pinot Saint-George, Primitivo di Gioa, Red Veltliner, Refosco, Rkatsiteli, Royalty, Rubired, Ruby Cabernet, Saint-Emilion, Saint Macaire, Salvador, Sangiovese, Sauvignon blanc, Sauvignon gris, Sauvignon vert, Scarlet, Seedless (including, e.g., red, black, white (green), purple seedless), Seibel 5279, Seibel 9110, Seibel 13053, Semillon, Servant, Shiraz (including, e.g., Brown and Green Shiraz), Souzao, Sultana Crimson, Sylvaner, Tannat, Teroldico, Tinta Madeira, Tinto cao, Touriga, Traminer, Trebbiano Toscano, Trousseau, Valdepenas, Viognier, Walschriesling, White Riesling, and Zinfandel.

Grape stems and vines are particularly advantageous for use in the present methods because such stems and vines are cheaply and readily available as waste from wine and juice-making processes. Typically, the grape berries are separated from their stems and vines before being pressed and the resulting juice fermented or stored. The remaining stems and vines are well-suited for extraction by the present methods. Fresh stems and vines, i.e., those picked less than 20 days before extraction according to the present invention, yield the most potent extracts. However, older stems and vines may still be used in the methods and compositions of the present invention.

It should be understood that the present methods do not include known methods of wine-making in which the grapes are pressed or fermented with their stems and vines. Such methods produce grape pomace which may include up to 30% stems by weight as well as seeds, skins, and pulp. However, the polyphenolic content of the pomace is less than that of an equivalent amount of fresh stems due to the extraction of the polyphenolic compounds into the grape juice/wine, and/or the decomposition of the polyphenolic compounds in the pomace.

In accordance with another aspect of the present invention, there are provided methods of extracting polyphenolic compounds from stems and vines, the method comprising treating a mixture comprising polyphenolic-containing stems, vines, or combinations thereof with one or more solvents to produce an extract containing polyphenolic compounds, wherein the extract comprises at least 40 mg gallic acid equivalents (GAE)/g-dry weight of extract. In some embodiments, the extract comprises at least 60 mg GAE/g-dry weight of extract or even at least 100 mg GAE/g-dry weight of extract. As used herein, the phrase “gallic acid equivalent” refers to an equivalent amount of calorimetric change for a given amount of gallic acid in the Folin-Ciocalteau assay, a common way of measuring total polyphenolics by assaying their redox potential.

In accordance with other aspects of the invention, there are provided compositions including the extracts produced by any of the methods described herein. The resulting extracts may be used in nutraceutical compositions, suitable for the addition to foods or beverages, or in dietary supplements in powder, pill or liquid form. The extracts may also be used in a pharmaceutical composition comprising the extract and a pharmaceutically acceptable carrier or diluent as described below.

A dietary supplement or a nutraceutical composition may be prepared comprising an extract produced as described herein and a food grade carrier. The weight ratio of the extract to carrier varies from about 0.01 to 100, and is more typically between about 0.1 and 100, about 0.1 and 10, or about 0.1 and 1. The extract may optionally be lyophilized (to produce a powder) prior to use in the composition. Nutraceutical compositions of the invention may further include food grade acids to prevent decomposition of the polyphenolics. Preferably the acids do not add flavor to the food; for example, ascorbic acid (vitamin C) is suitable for use in the present compositions.

As used herein, the term “carrier” refers to a composition which is added to increase the volume or bulk of a composition of the invention, but does not interfere with the activity of the active ingredients, i.e., polyphenolics, in the composition. Food grade carriers suitable for use include but are not limited to any edible starch or protein or materials containing starch or protein such as non-fat dry milk. Accordingly, flour, sugar, soybean meal, and maltodextrin may all be used as food grade carriers. Condiments such as salt, pepper, spices, herbs and the like may also serve as food grade carriers of the invention.

There are further provided compositions including combinations of inventive extract and other polyphenolic-containing extracts. For example, stem and vine extracts as described herein can be combined with grape seed extract, grape skin extract, or both grape seed and grape skin extract. The effects of the combination may be additive or, surprisingly, in some cases the effects may be synergistic (see Example 7). The ratio of inventive extract to other polyphenolic-containing extracts in combination compositions span a wide range and include from 0.01 to 100, or from 0.1, 0.25, or 0.5 to 1, 10, or 20.

Dietary supplements and nutraceutical compositions of the invention may be formulated in powder or liquid form by techniques well known in the art. Thus such techniques include those used for or similar to the techniques described below for the formulation of pharmaceutical compositions.

The composition is introduced into the food in an amount between about 0.1 and 10 mg/gm of the active ingredients of the food. The amount is preferably selected so as not to affect the taste of the food and to produce the most beneficial result. The food can be high (wet) or low moisture (dry) as is well known to those skilled in the art. When used as a dietary supplement the tablets contain between 0.1 to 1 gram or more of active ingredients. A particular food is cooked meat and other prepared foods where the composition provides antioxidant properties to the food and optionally color. The composition can be dispensed as a condiment on the prepared food to provide the nutraceutical benefits.

The instant invention also provides for pharmaceutical compositions which may be prepared by mixing stem and/or vine extract, pharmaceutically acceptable salts thereof, or solvates thereof, with pharmaceutically acceptable carriers, excipients, binders, diluents or the like to treat or ameliorate a variety of atherosclerotic disorders. The compositions of the inventions may be used to create formulations and prevent or treat coronary artery disease. Such compositions can be in the form of, for example, granules, powders, tablets, capsules, syrup, suppositories, injections, emulsions, elixirs, suspensions or solutions. The instant compositions can be formulated for various routes of administration, for example, by oral administration, by nasal administration, by rectal administration, subcutaneous injection, intravenous injection, intramuscular injections, or intraperitoneal injection. The following dosage forms are given by way of example and should not be construed as limiting the instant invention.

A “pharmaceutically acceptable salt” includes a salt with an inorganic base, organic base, inorganic acid, organic acid, or basic or acidic amino acid. As salts of inorganic bases, the invention includes, for example, alkali metals such as sodium or potassium; alkaline earth metals such as calcium and magnesium, aluminum; and ammonia. As salts of organic bases, the invention includes, for example, trimethylamine, triethylamine, pyridine, picoline, ethanolamine, diethanolamine, and triethanolamine. As salts of inorganic acids, the instant invention includes, for example, hydrochloric acid, hydroboric acid, nitric acid, sulfuric acid, and phosphoric acid. As salts of organic acids, the instant invention includes, for example, formic acid, acetic acid, lactic acid, trifluoroacetic acid, fumaric acid, oxalic acid, tartaric acid, maleic acid, citric acid, succinic acid, malic acid, methanesulfonic acid, benzenesulfonic acid, and p-toluenesulfonic acid. As salts of basic amino acids, the instant invention includes, for example, arginine, lysine and ornithine. Acidic amino acids include, for example, aspartic acid and glutamic acid.

For oral, buccal, and sublingual administration, powders, suspensions, granules, tablets, pills, capsules, gelcaps, and caplets are acceptable as solid dosage forms. These can be prepared, for example, by mixing one or more compounds of the instant invention, or pharmaceutically acceptable salts or tautomers thereof, with at least one additive such as a starch or other additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms can contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, binders, thickeners, buffers, sweeteners, flavoring agents or perfuming agents. Tablets and pills may be further treated with suitable coating materials known in the art.

Liquid dosage forms for oral administration may be in the form of pharmaceutically acceptable emulsions, syrups, elixirs, suspensions, and solutions, which may contain an inactive diluent, such as water. Pharmaceutical formulations and medicaments may be prepared as liquid suspensions or solutions using a sterile liquid, such as, but not limited to, an oil, water, an alcohol, and combinations of these. Pharmaceutically suitable surfactants, suspending agents, emulsifying agents, may be added for oral or parenteral administration.

As noted above, suspensions may include oils. Such oils include, but are not limited to, peanut oil, sesame oil, cottonseed oil, corn oil and olive oil. Suspension preparation may also contain esters of fatty acids such as ethyl oleate, isopropyl myristate, fatty acid glycerides and acetylated fatty acid glycerides. Suspension formulations may include alcohols, such as, but not limited to, ethanol, isopropyl alcohol, hexadecyl alcohol, glycerol and propylene glycol. Ethers, such as but not limited to, poly(ethyleneglycol), petroleum hydrocarbons such as mineral oil and petrolatum; and water may also be used in suspension formulations.

For nasal administration, the pharmaceutical formulations and medicaments may be a spray or aerosol containing an appropriate solvent(s) and optionally other compounds such as, but not limited to, stabilizers, antimicrobial agents, antioxidants, pH modifiers, surfactants, bioavailability modifiers and combinations of these. A propellant for an aerosol formulation may include compressed air, nitrogen, carbon dioxide, or a hydrocarbon based low boiling solvent.

Injectable dosage forms generally include aqueous suspensions or oil suspensions which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. Alternatively, sterile oils may be employed as solvents or suspending agents. Preferably, the oil or fatty acid is nonvolatile, including natural or synthetic oils, fatty acids, mono-, di- or tri-glycerides.

For injection, the pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

For rectal administration, the pharmaceutical formulations and medicaments may be in the form of a suppository, an ointment, an enema, a tablet or a cream for release of compound in the intestines, sigmoid flexure and/or rectum. Rectal suppositories are prepared by mixing one or more compounds of the instant invention, or pharmaceutically acceptable salts or tautomers of the compound, with acceptable vehicles, for example, cocoa butter or polyethylene glycol, which is present in a solid phase at normal storing temperatures, and present in a liquid phase at those temperatures suitable to release a drug inside the body, such as in the rectum. Oils may also be employed in the preparation of formulations of the soft gelatin type and suppositories. Water, saline, aqueous dextrose and related sugar solutions, and glycerols may be employed in the preparation of suspension formulations which may also contain suspending agents such as pectins, carbomers, methyl cellulose, hydroxypropyl cellulose or carboxymethyl cellulose, as well as buffers and preservatives.

Besides those representative dosage forms described above, pharmaceutically acceptable excipients and carriers are generally known to those skilled in the art and are thus included in the instant invention. Such excipients and carriers are described, for example, in “Remingtons Pharmaceutical Sciences” Mack Pub. Co., New Jersey (1991), which is incorporated herein by reference.

The formulations of the invention may be designed to be short-acting, fast-releasing, long-acting, and sustained-releasing as described below. Thus, the pharmaceutical formulations may also be formulated for controlled release or for slow release.

The instant compositions may also comprise, for example, micelles or liposomes, or some other encapsulated form, or may be administered in an extended release form to provide a prolonged storage and/or delivery effect. Therefore, the pharmaceutical formulations and medicaments may be compressed into pellets or cylinders and implanted intramuscularly or subcutaneously as depot injections or as implants such as stents. Such implants may employ known inert materials such as silicones and biodegradable polymers.

In accordance with another aspect of the invention, the extracts of the invention may be used in the prevention and treatment of heart disease. In particular, the invention provides in some aspects methods of preventing or treating coronary artery disease, cerebro-vascular disease, peripheral-vascular disease, atherosclerosis, atherosclerosis-related disease, or heart failure including administering to a subject in need thereof an effective amount of the extract as described herein. “Treating” within this context, means an alleviation, in whole or in part, of symptoms associated with a disorder or disease, or halt of further progression or worsening of those symptoms, or prevention or prophylaxis of the disease or disorder. Similarly, as used herein, an “effective amount” of an inventive extract refers to an amount of the extract that alleviates, in whole or in part, symptoms associated with a disorder or disease, or halts of further progression or worsening of those symptoms, or prevents or provides prophylaxis for the disease or disorder.

In some embodiments, the extract is administered post-prandially, i.e., with food or within about 1 or 2 hours of eating. An effective amount of extract may typically be produced from 10-1000 mg stems, vines, leaves or combinations thereof/kg of body weight. More typically, an effective amount of extract is the extract produced from 50-250 mg stems, vines, leaves or combinations thereof/kg of body weight. Hence, in some embodiments, an effective amount of extract ranges from about 1 to about 50 mg extract (dry weight)/kg body weight.

An effective amount of a compound of the present invention may vary depending upon the route of administration and dosage form. Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant invention.

Compositions of the invention can be administered to any animal that can experience the beneficial effects of the compounds of the invention. Typically, the animal is a mammal, and in particular a human, although the invention is not intended to be so limited. The term “subject” as used herein therefore means any animal that can experience the beneficial effects of the compounds of the invention.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member of the range.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are provided in the following examples by way of illustration and are not intended to be limiting of the present invention. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

The following definitions are used throughout:

| | |
|---|---|
| ADP | Adenosine diphosphate |
| CFR | Cyclic flow reduction |
| DMSO | Dimethylsulfoxide |
| EDTA | N,N-ethylenediamine tetraacetic acid |
| GAE | Gallic Acid Equivalents |
| LDL | Low density lipoproteins |
| mL | Milliliter(s) |
| mM | Millimolar |
| μg | Microgram(s) |
| μL | Microliter(s) |
| μM | Micromolar |
| nm | Nanometer(s) |
| PMA | Phorbol-12-myristate-13-acetate |

### Example 1

### Extraction of Stems

Procedure I: Approximately 5 grams of fresh grape stem is frozen in liquid nitrogen and ground in a high-speed laboratory mill to produce a fine powder. The ground material is suspended in 25 mL of 80% aqueous acetone and placed in an ultrasonic bath for 10 minutes. The mixture is centrifuged and the supernatant removed. The remaining residue is extracted three more times by the same procedure and the combined organic fractions are concentrated under reduced pressure while raising the temperature slightly. The resulting aqueous solutions are either dried further or are diluted with DMSO and saline to provide a water/DMSO saline solution. The same procedure can be used to produce grape seed and grape skin extracts for comparison.

To compare extract potency, solutions from the above extraction were subjected to a Folin-Ciocalteau assay to measure polyphenolic content in terms of GAE as described by Singleton and Rossi in the American Journal of Enology and Viticulture (1965; 16: 144-158). As shown in Table 1 below, on a dry weight basis grape stems (Concord) have a significantly higher proportion of GAE than pulp, skin or seed.

TABLE 1

| | | Average Dry | | Total GAE | |
|---|---|---|---|---|---|
| | Total Weight | Weight (% of | Dry Weight | Recovered | GAE/Dry Wt |
| Component | (g) | Total) | (g) | (mg) | (mg/g) |
| Pulp | 4.94 | 16.59 | 0.819 | 1.58 | 1.93 |
| Skin | 5.19 | 24.37 | 1.265 | 46.03 | 36.40 |
| Seed | 4.94 | 70.31 | 3.473 | 108.87 | 31.34 |
| Stem | 5.19 | 28.08 | 1.457 | 95.36 | 65.43 |
| Pulp/Skin | 5.20 | 16.53 | 0.860 | 8.92 | 10.38 |

Procedure II: Thirty grams of stems or stems and vines are separated from about 2-3 bunches of fresh grapes. The stems are thoroughly frozen in liquid nitrogen and ground into a powder using a suitable grinder. Aqueous acetone (80% acetone by volume; water is pH 7), is added to the stem powder at the rate of 20 mL solvent per gram of stem powder. This mixture is sonicated for 40 minutes to provide a liquid extract. The latter extract is centrifuged at 2500 RPM (1280×g) for 10 minutes at 10° C. to pellet particulate mater such as insoluble fibrous components. The supernatant is transferred to a rotary evaporator and the acetone removed under reduced pressure. The acetone-free extract is chromatographed on a Sephadex LH-20 column using a step gradient (i.e., the column is washed with several volumes of water (pH 7), followed by elution of the polyphenolics with 80% acetone/water (water is pH 7). The polyphenolic-containing fractions are concentrated under reduced pressure to remove acetone and then are lyophilized to yield a grape stem powder extract, suitable for further studies.

Grape stem extracts from seven different grape varieties produced according to Procedure II were tested for total polyphenolic content using the Folin-Ciocalteau method as described above. Results are shown in Table 2 below. Brown Shiraz and Red Globe appear to have significantly more polyphenolic content than the other varieties tested, but all showed good amounts. A mass spectroscopic analysis (MALDI-TOF) of the extract according to the procedure of Krueger, C. G. et al. (J. Agric. Food Chem. 48, 1663-67, 2000) showed that the grape stem extract contained similar or greater quantities of polygalloyl flavan-3-ols compared to grape seed and grape skin extract.

TABLE 2

| | | % Inhibition of |
|---|---|---|
| | mg GAE/mL of | Collagen-Induced |
| Grape Variety | Liquid Extract | Platelet Aggregation |
| Green Shiraz | 8.99 | 50.8 |
| Brown Shiraz | 13.11 | 52.9 |
| Concord | 7.17 | 50.7 |
| Green Seedless | 7.2 | 64.3 |
| Black Seedless | 7.7 | 73.7 |
| Red Globe | 13.68 | 70.7 |
| Red Seedless | 8.25 | 38.3 |

### Example 2

### In Vitro Platelet Aggregation

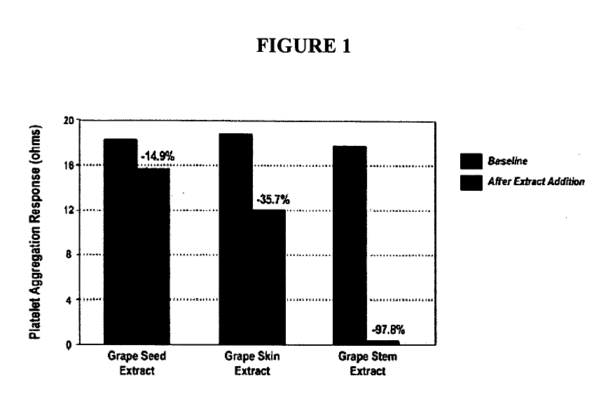

Platelet Aggregometry was performed according to the method of Shanmuganayagam et al. in Methods of Enzymology (2001; 335: 369-380). Human blood was diluted with PF Saline to form a 50% solution. One mL of the diluted blood was placed in a cuvette containing a stir bar. The cuvette was placed in the aggregometer where it was stirred and heated to 37° C. Dry weight normalized test extract (14 μL) or control (0 μL) was added to the cuvette. Electrodes were inserted into the cuvette and the resistance of the blood sample measured over time after addition of a platelet agonist (2 μg collagen). FIG. 1 shows the platelet aggregation response of grape stem extract (Vitis vinerfera) prepared according to procedure I versus calculated dry weight equivalent amounts of grape skin extract and grape seed extract. This example shows that on a per weight basis grape stem extract is a far more potent platelet aggregation inhibitor than grape skin or grape seed extract.

The same method of measuring platelet aggregation was used on a variety of extracts prepared according to procedure II. Results are shown in Table 2, above. Stem extracts from each grape varietal displayed significant inhibition of collagen-induced platelet aggregation. Extracts from black seedless and red globe varietals are the most potent in this assay.

### Example 3

### Effect of Extracts in the Cyclic Flow Reduction Model of Coronary Artery Disease

In this example the effect of grape stem extract on cyclic flow reductions was examined according to the method of Folts (Circulation 1991; 83 (suppl IV): IV-3-IV-14; Circulation 1995, 91: 1182-1188). The method was generally performed as follows. The animal is anesthetized with a barbiturate (e.g., 20 mg/kg sodium pentobarbital) and may be pre-medicated first with, e.g., either 3 mg/kg morphine sulfate (dogs) or 20 mg/kg ketamine i.m. (cynomologous monkey). The animal is mechanically ventilated and the chest surgically opened to expose the heart. A major branch of the coronary artery is dissected out. A circumflex coronary artery flow probe is placed on the dissected coronary artery. A section of the artery downstream to the flow probe is clamped with a vascular clamp to cause damage to the arterial wall. A plastic cylinder is placed around the outside of the artery to narrow the arterial lumen by 50% to 60%. Blood platelets begin to accumulate in the damaged, narrowed artery and form a blood clot. As the clot grows larger, the flow probe detects the decline in coronary blood flow. The plastic cylinder is gently shaken to dislodge the blood clot and restore blood flow through the artery. Once the clot is dislodged another clot begins to form causing a decline in the blood flow once again. These periodic reductions in coronary blood flow are called cyclic flow reductions or CFRs. These have been observed in patients with coronary artery disease and are the cause of unstable angina. Administration of effective platelet inhibitors to the animal can abolish the CFRs.

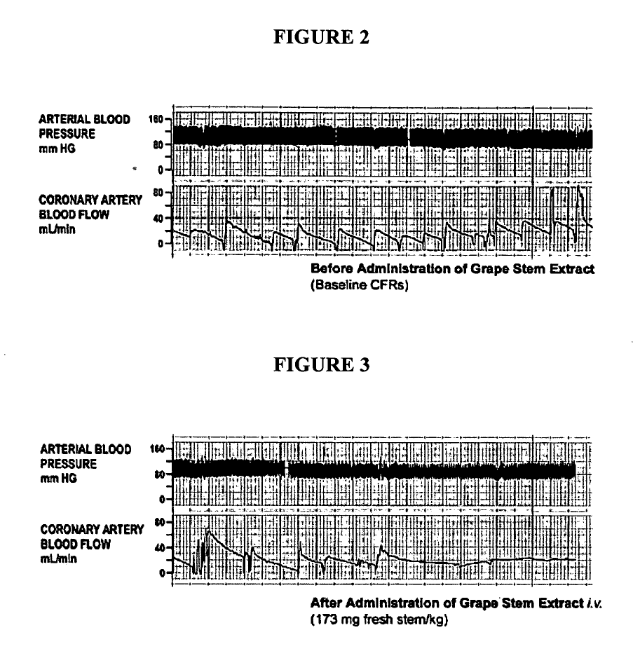

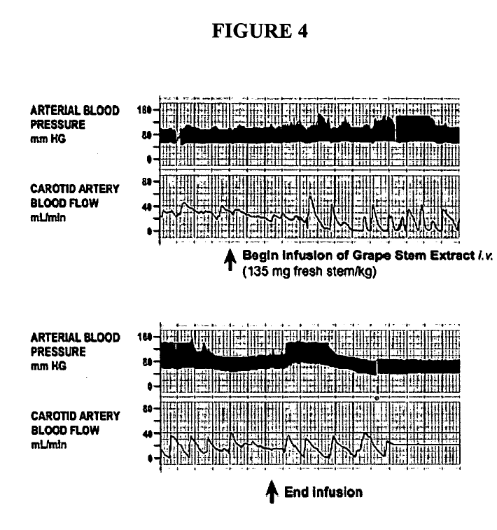

Grape stem extract of the present invention was tested in the canine CFR model. FIG. 2 shows the baseline CFRs before administration of grape stem extract. FIG. 3 shows that the IV administration of extract from 173 mg fresh stem/kg body weight to a dog abolished the CFRs. Likewise FIG. 4 shows the effect of grape stem extract administration on carotid artery thrombosis in the monkey CFR model. Administration of extract from 135 mg fresh stem/kg abolished the CFRs. The abolishment of the CFRs is an indication that an effective platelet inhibitor has been administered.

### Example 4

### Ex-Vivo Platelet Aggregation Response to Infusion of Grape Stem Extract in Cyclic Flow Reduction Models

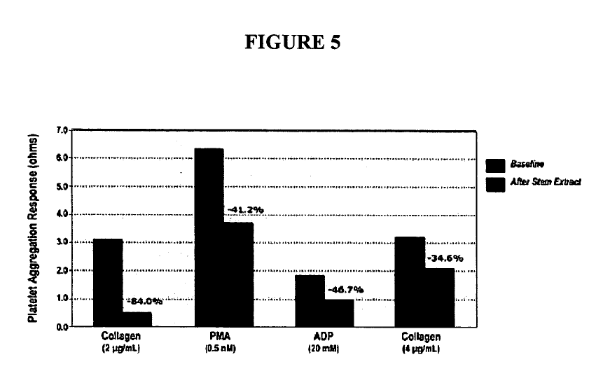

Platelet aggregation was measured according to the procedure of Example 2 on blood drawn before and after the administration of grape stem extract (Procedure I, Example I) to the canine of Example 3. Aggregation was induced using collagen (2 μg/mL and 4 μg/mL), PMA (0.5 nM), and ADP (20 mM), as platelet agonists. As shown in FIG. 5, administration of extract from 173 mg fresh stem/kg per dog significantly inhibited platelet aggregation induced by each of the agonists.

### Example 5

### Antioxidant Effect

Extracts of grape stem, skin and seed (Vitis vinifera, Red Globe) prepared as in Procedure II, Example 1 were compared to control for their ability to inhibit LDL oxidation. For comparison, antioxidant activity of grape seed and grape skin extract used in PROVEX CV (MELALEUCA, Inc.) was assessed. (These extracts are among the most potent grape seed/skin extracts that are commercially available.) LDL was isolated from human plasma using density gradient ultracentrifugation utilizing OPTIPREP (Iodoxinol) to form a density gradient according to the manufacturer's directions. (See OPTIPREP application sheet M-11.) The assay was performed in a 96 well microtiter plate by modification of known methods. (Kleinfeld et al. Clin. Chem. (1992) 38, 2066-72 and Cole et al. Clin. Chem. (1999) 45, 696-99.) Each well contained: 50 μL diluted calculated dry weight equivalent amounts of seed, skin or stem extract, 1 μM EDTA, 5 μM Cu²⁺, and 0.05 mg/mL LDL protein. Once the wells were loaded, the plate was placed in a reader at 30° C. and the absorbance was read at 234 nm every 3 minutes for 10 hours. The plate was shaken for 15 seconds prior to each measurement.

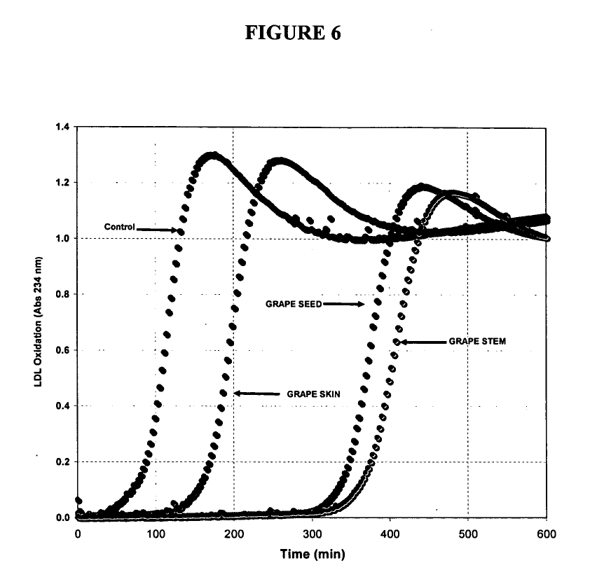

FIG. 6 shows a graph of absorbance versus time; absorbance increases as LDL oxidation takes place. The figure shows that LDL begins to significantly oxidize after about 1½ hours in the absence of any antioxidant. By comparison, extracts of grape skin and grape seed and grape stem delay the onset of oxidation. These data show that grape stem extract exhibits antioxidant activity similar to or better than that of grape seed and skin extract.

### Example 6

### Inhibition of In Vitro Platelet Aggregation by Combinations of Grape Stem Extracts and Other Polyphenolic Extracts

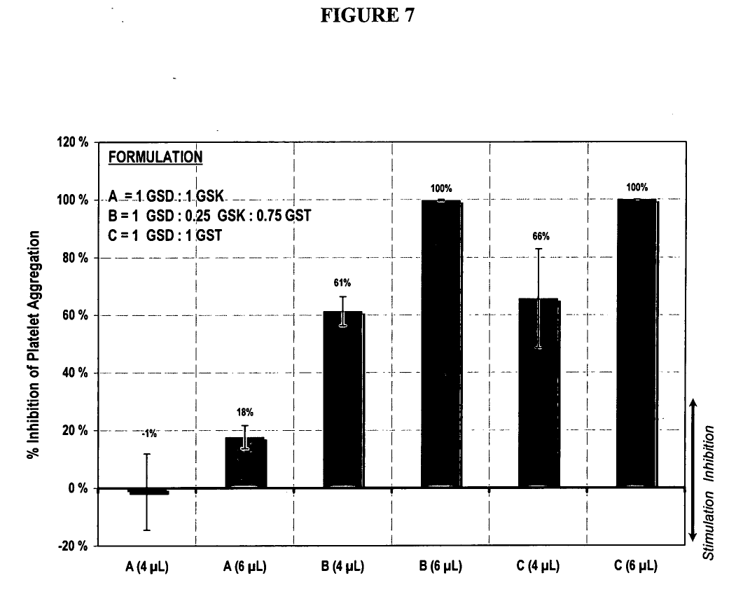

Combinations of grape stem extract (Red Globe prepared according to Procedure II, Example 1) with grape seed and grape skin were assessed for anti-platelet activity in human blood according to the procedure of Example 2. Sample A is PROVEX CV (MELALEUCA, Inc.), a 1:1 mixture of grape seed extract (GSD) to grape skin extract (GSK). Sample B is a 1:0.25:0.75 mixture of GSD, GSK, and grape stem extract (GST) prepared according to Procedure II, Example 1. Sample C is a 1:1 mixture of GSD and GST. Two concentrations of each sample were tested. Results are shown in FIG. 7. The results show that replacement of part (B) or all (C) of the polyphenols from grape skin extract with those of grape stem extract has a synergistic effect with grape seed extract on the inhibition of platelet aggregation. Hence, grape stem extract may improve potency of commercially available grape seed extracts at reduced cost.

Although the foregoing refers to particular illustrative embodiments, it will be understood that the present invention is not so limited. It will occur to those of ordinary skill in the art that various modifications may be made to the disclosed embodiments and that such modifications are intended to be within the scope of the present invention, which is defined by the following claims.

1. A method comprising
treating a mixture comprising polyphenolic-containing stems, vines, or combinations thereof with one or more solvents to produce an extract containing polyphenolic compounds; and
enriching the extract in polyphenolic compounds by chromatography.

2. The method of claim 1 further comprising processing the mixture to increase the surface area of the stems, vines, or combinations thereof prior to treating with one or more solvents.

3. The method of claim 1 further comprising freezing the stems, vines or combinations thereof and grinding the frozen stems, vines, or combinations thereof prior to treating with one or more solvents.

4. The method of claim 1, wherein the mixture is treated with one or more solvents two or more times to produce the extract.

5. The method of claim 1, wherein the one or more solvents is selected from the group consisting of water, alcohols, esters, sulfoxides, ketones, and mixtures of any two or more thereof.

6. The method of claim 1, wherein the one or more solvents is selected from the group consisting of water, methanol, ethanol, ethyl acetate, t-butyl acetate, dimethylsulfoxide, acetone, methylethylketone, and mixtures of any two or more thereof.

7. The method of claim 1, wherein the one or more solvents is selected from the group consisting of water/acetone, water/methanol, water/ethanol, and water/DMSO.

8. The method of claim 7, wherein the water has a pH of about 2 to about 8.

9. The method of claim 7, wherein the one or more solvents is a mixture of acetone and water.

10. The method of claim 9, wherein the mixture of acetone and water is from about 50% acetone to about 99% acetone by volume.

11. The method of claim 1 further comprising removing insoluble material from the extract by centrifugation or filtration.

12. The method of claim 11, wherein the filtration is microfiltration or ultrafiltration.

13. The method of claim 1, wherein the chromatography is adsorption/permeation chromatography.

14. The method of claim 1, wherein the chromatography is conducted using a solvent gradient.

15. The method of claim 14, wherein the solvent gradient is a step gradient.

16. The method of claim 14, wherein the solvent gradient runs from a polar solvent to a less polar solvent.

17. The method of claim 15, wherein the polar solvent is water and the less polar solvent is a mixture of water and acetone.

18. The method of claim 1, wherein the mixture further comprises polyphenolic-containing leaves.

19. The method of claim 1, wherein the polyphenolic containing stems or vines are grape, cranberry, or blueberry stems or vines.

20. The method of claim 1, wherein the stems or vines are grape stems or vines.

21. The method of claim 20 wherein the grape stems or vines are selected from the group consisting of Aleatico, Alicante Bouschet, Aligote, Alvarelhao, Aramon, Baco blanc, Burger, Cabernet franc, Cabernet, Sauvignon, Calzin, Carignane, Charbono, Chardonnay, Chasselas dore, Chenin blanc, Clairette blanche, Concord, Early Burgundy, Emerald Riesling, Feher Szagos, Femao Pires, Flora, French Colombard, Fresia, Furnint, Gamay, Gewurztraminer, Grand noir, Gray Riesling, Green Hungarian, Green Veltliner, Grenache, Grillo, Helena, Inzolia, Lagrein, Lambrusco de Salamino, Malbec, Malvasia bianca, Mataro, Melon, Merlot, Meunier, Mission, Montua de Pilas, Muscadelle du Bordelais, Muscat blanc, Muscat Ottonel, Muscat Saint-Vallier, Nebbiolo, Nebbiolo fino, Nebbiolo Lampia, Orange Muscat, Palomino, Pedro Ximenes, Petit Bouschet, Petite Sirah, Peverella, Pinot noir, Pinot Saint-George, Primitivo di Gioa, Red Veltliner, Refosco, Rkatsiteli, Royalty, Rubired, Ruby Cabernet, Saint-Emilion, Saint Macaire, Salvador, Sangiovese, Sauvignon blanc, Sauvignon gris, Sauvignon vert, Scarlet, Seedless, Seibel 5279, Seibel 9110, Seibel 13053, Semillon, Servant, Shiraz, Souzao, Sultana Crimson, Sylvaner, Tannat, Teroldico, Tinta Madeira, Tinto cao, Touriga, Traminer, Trebbiano Toscano, Trousseau, Valdepenas, Viognier, Walschriesling, White Riesling, Zinfandel, and combinations thereof.

22. The method of claim 20, wherein the grape stems or vines are green shiraz, brown shiraz, concord, green seedless, black seedless, red globe, red seedless or combinations thereof.

23. The method of claim 20, wherein the grape stems or vines are black seedless or red globe grapes.

24. The extract produced by the method of claim 1.

25. The extract of claim 24, wherein the extract is enriched in polygalloyl polyflavan-3-ols by the chromatography step.

26. The composition of claim 24 further comprising grape seed extract, grape skin extract, or both.

27. A pharmaceutical composition comprising the extract of claim 24 and a pharmaceutically acceptable carrier or diluent.

28. A dietary supplement or a nutraceutical composition comprising the extract of claim 24 and a food grade carrier.

29. The dietary supplement or a nutraceutical composition of claim 28 further comprising a food grade acid.

30. The dietary supplement or a nutraceutical composition of claim 28 further comprising grape seed extract, grape skin extract, or both.

31. A method of preventing or treating coronary artery disease, cerebro-vascular disease, peripheral-vascular disease, atherosclerosis, atherosclerosis-related disease, or heart failure comprising administering to a subject in need thereof an effective amount of the extract of claim 24.

32. The method of claim 31, wherein the extract is administered with food or within 2 hours of eating.

33. A method of preventing or treating coronary artery disease, cerebro-vascular disease, peripheral-vascular disease, atherosclerosis, atherosclerosis-related disease, or heart failure comprising administering to a subject in need thereof an effective amount of the extract of claim 25.

34. A method of extracting polyphenolic compounds from stems and vines, the method comprising treating a mixture comprising polyphenolic-containing stems, vines, or combinations thereof with a mixture of water and acetone to produce an extract containing polyphenolic compounds.

35. The method of claim 34, wherein the mixture of acetone and water ranges from about 50% acetone to about 99% acetone by volume.

36. The method of claim 34 further comprising enriching the extract in polyphenolic compounds by chromatography.

37. The method of claim 34 wherein the chromatography is adsorption/permeation chromatography.

38. An extract of polyphenolic-containing stems and vines produced by the method of claim 34.
